(19) United States
(12) Patent Application Publication
(10) Pub. No.: US 2021/0220312 A1
(43) Pub. Date: Jul. 22, 2021

(54) Compositions And Methods For Modulating Cancer

(71) Applicant: Tyme, Inc., Bedminster, NJ (US)

(72) Inventor: Steven Hoffman, Mahwah, NJ (US)

(21) Appl. No.: 17/150,000

(22) Filed: Jan. 15, 2021

Related U.S. Application Data
(60) Provisional application No. 62/962,523, filed on Jan. 17, 2020.

(51) Int. Cl. A61K 31/198; A61K 45/06; A61K 31/4166; A61K 31/55; A61K 31/436; A61P 35/04

(52) U.S. Cl. CPC A61K 31/198; A61K 45/06; A61P 35/04; A61K 31/55; A61K 31/436; A61K 31/4166

(57) ABSTRACT

The present inventions provide compositions and methods for modulating cancer using a tyrosine derivative.

### CROSS-REFERENCE TO RELATED APPLICATION

This application claims the benefit of priority to U.S. Provisional patent Application No. 62/962,523, filed Jan. 17, 2020, the entirety of which is incorporated by reference herein.

### TECHNICAL FIELD

The present inventions relate generally to methods for modulating cancer, and in particular for inhibiting tumor growth and metastasis.

### BACKGROUND

According to the U.S. National Cancer Institute's Surveillance Epidemiology and End Results (SEER) database for the year 2008, 11,958,000 Americans have invasive cancers. Cancer is the second most common cause of death in the United States, behind only heart disease, and accounts for one in four deaths. It has been estimated that approximately 1600 Americans die of cancer each day. In addition to the medical, emotional and psychological costs of cancer, cancer has significant financial costs to both the individual and society. It is estimated by the National Institutes of Health that the overall costs of cancer in 2010 was $263.8 billion. In addition, it is estimated that another $140.1 billion is lost in productivity due to premature death.

Cancer treatments today include surgery, hormone therapy, radiation, chemotherapy, immunotherapy, targeted therapy, and combinations thereof. Surgical removal of cancer has advanced significantly; however, there remains a high chance of recurrence of the disease. Hormone therapy using drugs such as aromatase inhibitors and luteinizing hormone-releasing hormone analogs and inhibitors has been relatively effective in treating prostate and breast cancers. Radiation and the related techniques of conformal proton beam radiation therapy, stereotactic radiosurgery, stereotactic radiation therapy, intraoperative radiation therapy, chemical modifiers, and radio sensitizers are effective at killing cancerous cells, but can also kill and alter surrounding normal tissue. Chemotherapy drugs such as aminopterin, cisplatin, methotrexate, doxorubicin, daunorubicin and others alone and in combinations are effective at killing cancer cells, often by altering the DNA replication process. Biological response modifier (BRM) therapy, biologic therapy, biotherapy, or immunotherapy alter cancer cell growth or influence the natural immune response, and involve administering biologic agents to a patient such as an interferons, interleukins, and other cytokines and antibodies such as rituximab and trastuzumab and even cancer vaccines such as Sipuleucel-T.

Targeted therapies also have been developed to fight cancer. These targeted therapies differ from chemotherapy because chemotherapy works by killing both cancerous and normal cells, with greater effects on the cancerous cells. Targeted therapies work by influencing the processes that control growth, division, and the spread of cancer cells and signals that cause cancer cells to die naturally. One type of targeted therapy includes growth signal inhibitors such as trastuzumab, gefitinib, imatinib, centuximab, dasatinib and nilotinib. Another type of targeted therapy includes angiogenesis inhibitors such as bevacizumab that inhibit cancers from increasing surrounding vasculature and blood supply. A final type of targeted therapy includes apoptosis-inducing drugs that are able to induce direct cancer cell death.

None of the treatments mentioned above inhibits cancer development. Generally speaking, development of cancer is a multistep process by which cells acquire genetic mutations, lose the ability to control proliferation, invade surrounding tissues and metastasize. This process, sometimes referred to as carcinogenesis, has been characterized as proceeding through four stages: initiation, promotion, progression, and conversion. See, e.g., McKinnell, R. G. et al., The Biological Basis of Cancer, Cambridge: Cambridge University Press, 1998, pages 79-81.

Initiation refers to the acquisition of a genetic mutation. Genetic mutations can be caused by exposure of a cell to, for example, carcinogens (e.g. certain chemicals, ionizing radiation), or exposure to certain viruses.

In the promotion stage, initiated cells proliferate to form benign tumors or hyperplastic lesions.

In the progression stage, the cells acquire additional genetic mutations, such as through carcinogen exposure, which allow the cells to express a neoplastic phenotype.

Conversion, or malignant transformation, refers to the acquisition of the malignant phenotype, by which the transformed cells invade surround tissues and spread.

There is a great unmet need for methods to slow or halt the carcinogenesis process, and thereby slow or halt the conversion of precancerous cells into malignant cancer. Slowing the carcinogenesis process would provide more time for the application of traditional therapeutic interventions.

### SUMMARY

The present invention provides methods of reducing the rate of tumor growth in a subject, comprising administering to the subject an effective amount of a tyrosine derivative.

The present invention also provides methods of inhibiting malignant transformation of precancerous cells in a subject comprising administering to said subject an effective amount of a tyrosine derivative.

The present invention also provides methods of inhibiting cancer metastasis in a subject comprising administering to said subject and an effective amount of a tyrosine derivative.

The present invention also provides methods of reducing the number of circulating metastatic seed cells in a subject, comprising administering to the subject an effective amount of a tyrosine derivative.

The present invention also provides methods of reducing the risk of developing cancer in a subject at increased risk of developing cancer as a result of exposure to a carcinogen or an oncovirus, comprising administering to said subject an effective amount of a tyrosine derivative.

### BRIEF DESCRIPTION OF THE DRAWINGS

FIG. 1 shows the HCT-116 tumor volume (y-axis; mm³, group average) vs. time (x-axis; days) in treated and untreated groups. The HCT116 tumor bearing athymic nude mice were PO dosed daily for 29 days at 5 ml/kg with water (Group 1), α-methyl-DL-tyrosine at 81 mg/kg (Group 2), α-methyl-DL-tyrosine at 162 mg/kg (Group 3), and α-methyl-DL-tyrosine at 324 mg/kg (Group 4).

### DETAILED DESCRIPTION OF ILLUSTRATIVE EMBODIMENTS

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As employed above and throughout the disclosure the term “effective amount” refers to an amount effective, at dosages, and for periods of time necessary, to achieve the desired result with respect to the relevant disorder, condition, or side effect. It will be appreciated that the effective amount of components of the present invention will vary from subject to subject not only with the particular compound, component or composition selected, the route of administration, and the ability of the components to elicit a desired result in the individual, but also with factors such as the disease state or severity of the condition to be alleviated, hormone levels, age, sex, weight of the individual, the state of being of the subject, and the severity of the pathological condition, concurrent medication or special diets then being followed by the particular subject, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The term “administering” means either directly administering a compound or composition of the present invention, or administering a prodrug, derivative or analog which will form an equivalent amount of the active compound or substance within the body.

The terms “subject,” “individual,” and “patient” are used interchangeably herein, and refer to an animal, for example a human. The term “subject” as used herein refers to human and non-human animals.

In some aspects, the present invention provides methods of reducing the rate of tumor growth in a subject, comprising administering to the subject an effective amount of a tyrosine derivative.

In these aspects, an effective amount of a tyrosine derivative is an amount effective to reduce the rate of tumor growth.

As used herein, the term “reducing the rate of tumor growth” refers to decreasing the rate at which the tumor increases in size. The rate at which a tumor is growing can be determined by measuring the size of the tumor over time, and thereby determining the change in tumor size as a function of time. Administration of a tyrosine derivative has reduced the rate of tumor growth when the change in tumor size as a function of time is less after administration of the tyrosine derivative than the change in tumor size as a function of time in the absence of administration of a tyrosine derivative.

In some embodiments, the tumor is a benign tumor; i.e., a non-malignant tumor.

In some embodiments, the benign tumor is a mass of precancerous cells.

In some embodiments, the benign tumor is a mass of precancerous skin cells.

In some embodiments, the benign tumor is a mass of precancerous cells of the respiratory tract, preferably, cells of the nasal cavity, paranasal sinuses, pharynx, larynx; trachea, bronchi, bronchioles, or lungs.

In some embodiments, the benign tumor is a mass of precancerous cells of the digestive tract, preferably, cells from mouth, throat; esophagus; stomach; small intestine; colon; rectum, or anus.

In some embodiments, the benign tumor is a mass of precancerous cells of the genitourinary tract organs, preferably, cells from kidney, bladder, prostate, testicles, uterus, fallopian tubes, ovaries, vagina, or external genitalia.

In some embodiments, the benign tumor is a mass of precancerous blood cells.

In some embodiments, the benign tumor is a mass of precancerous breast cells.

In some embodiments, the benign tumor is a mass of precancerous cells of the endocrine organs, preferably, cells of the hypothalamus; pineal gland; pituitary gland; thyroid; parathyroid; thymus; adrenal gland, or pancreas.

In some embodiments, the benign tumor is a mass of precancerous brain cells.

In some embodiments, the benign tumor that is a mass of precancerous cells is actinic keratosis (abnormal changes in the skin which may develop into squamous cell skin cancer); Bowen's disease (intraepidermal carcinoma/squamous carcinoma in situ); dyskeratosis congenita; ductal carcinoma in situ (breast); Sclerosing adenosis (breast); Small duct papilloma (breast); atypical lobular hyperplasia (which may develop into breast cancer); oral submucous fibrosis; erythroplakia; lichen planus (oral); leukoplakia; proliferative verrucous leukoplakia; stomatitis nicotina; Barrett's esophagus; atrophic gastritis (precancerous changes in the stomach which may develop into gastric (stomach) cancer; adenomatous polyps in the colon (which may develop into colon cancer); Plummer-Vinson syndrome (sideropenic dysphagia); hereditary nonpolyposis colorectal cancer (Lynch Syndrome); cervical dysplasia (cervical intraepithelial neoplasm, CIN); vaginal intraepithelial neoplasm (VAIN); anal dysplasia; lichen sclerosus; Bowen's disease (penile or vulvar); erythroplasia of Queyrat; bladder carcinoma in situ; monoclonal gammopathy of unknown significance; dysplastic moles (which may develop into melanoma); bronchial epithelial dysplasia (which may develop into lung cancer); multiple endocrine neoplasia type 1, multiple endocrine neoplasia type 2.

In other embodiments, the tumor is malignant tumor.

In some embodiments, the malignant tumor is non-small cell lung cancer brain cancer, appendix cancer, biliary cancer, choleangiocarcinoma, colon cancer, germ cell tumor, glioma, neuroblastoma, prostate cancer, tongue cancer, tonsil squamous cell carcinoma, urothelial cancer, adenoid cystic carcinoma, adrenal gland tumor, amyloidosis, anal cancer, ataxia-telangiectasia, Beckwith Wiedemann syndrome, bile duct cancer, Birt Hogg Dube syndrome, bladder cancer, bone cancer, brain tumor, breast cancer, breast cancer in men, carcinoid tumor, carney complex, cervical cancer, colorectal cancer, ductal carcinoma, endometrial cancer, esophageal cancer, gastric cancer, gastrontestinal stromal tumor—GIST, HER2-positive breast cancer, hereditary prostate cancer, islet cell tumor, juvenile polyposis syndrome, kidney cancer, laryngeal cancer, liver cancer, lobular carcinoma, lung cancer, lung cancer—small cell, malignant glioma, mastocytosis, melanoma, meningioma, multiple myeloma, myelodysplastic syndrome (MDS), nasopharyngeal cancer, neuroendocrine tumor, nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma (childhood), polycythemia vera, prostate cancer, renal cell cancer, retinoblastoma (childhood), salivary gland cancer, sarcoma, sarcoma—alveolar soft part and cardiac, sarcoma—Kaposi, skin cancer (non-melanoma), small bowel cancer, small intestine cancer, stomach cancer, testicular cancer, thymoma, thyroid cancer, Turcot syndrome, uterine (endometrial) cancer, vaginal cancer, Von-Hippel-Lindau syndrome, or Wilms' tumor (childhood).

In some aspects, the present invention provides methods of inhibiting malignant transformation of precancerous cells in a subject comprising administering to said subject an effective amount of a tyrosine derivative.

In these aspects, an effective amount of a tyrosine derivative is an amount effective to inhibit malignant transformation of precancerous cells.

As used herein, the term “inhibiting malignant transformation” refers to reducing the rate at which precancerous cells acquire a malignant phenotype. The rate at which precancerous cells acquire a malignant phenotype can be determined by measuring the population average rate of progression from observation of the precancerous cells to malignant transformation of those cells.

As used herein, the term “malignant transformation” refers to the acquisition by cells of a malignant phenotype. A malignant phenotype is characterized by the ability of a cell to invade surrounding tissues and metastasize.

As used herein, “precancerous cells” refer to abnormal cells that are at increased risk of undergoing malignant transformation (i.e., of becoming cancerous cells). Precancerous cells are sometimes referred to as precancerous lesions.

In some embodiments of the methods of the invention, the precancerous cells are skin cells.

In other embodiments, the precancerous cells are cells of the respiratory tract, including, for example, cells of the nasal cavity, paranasal sinuses, pharynx, larynx; trachea, bronchi, bronchioles, or lungs.

In other embodiments, the precancerous cells are cells of the digestive tract, including for example, cells from mouth, throat; esophagus; stomach; small intestine; colon; rectum, or anus.

In other embodiments, the precancerous cells are cells of the genitourinary tract organs, including for example, cells from kidney, bladder, prostate, testicles, uterus, fallopian tubes, ovaries, vagina, or external genitalia.

In other embodiments, the precancerous cells are blood cells.

In other embodiments, the precancerous cells are breast cells.

In other embodiments, the precancerous cells are cells of the endocrine organs, including for example, cells of the hypothalamus; pineal gland; pituitary gland; thyroid; parathyroid; thymus; adrenal gland, or pancreas.

In other embodiments, the precancerous cells are brain cells.

In some embodiments, the precancerous cells are actinic keratosis (abnormal changes in the skin which may develop into squamous cell skin cancer); Bowen's disease (intraepidermal carcinoma/squamous carcinoma in situ); dyskeratosis congenita; ductal carcinoma in situ (breast); Sclerosing adenosis (breast); Small duct papilloma (breast); atypical lobular hyperplasia (which may develop into breast cancer); oral submucous fibrosis; erythroplakia; lichen planus (oral); leukoplakia; proliferative verrucous leukoplakia; stomatitis nicotina; Barrett's esophagus; atrophic gastritis (precancerous changes in the stomach which may develop into gastric (stomach) cancer; adenomatous polyps in the colon (which may develop into colon cancer); Plummer-Vinson syndrome (sideropenic dysphagia); hereditary nonpolyposis colorectal cancer (Lynch Syndrome); cervical dysplasia (cervical intraepithelial neoplasm, CIN); vaginal intraepithelial neoplasm (VAIN); anal dysplasia; lichen sclerosus; Bowen's disease (penile or vulvar); erythroplasia of Queyrat; bladder carcinoma in situ; monoclonal gammopathy of unknown significance; dysplastic moles (which may develop into melanoma); bronchial epithelial dysplasia (which may develop into lung cancer); multiple endocrine neoplasia type 1, multiple endocrine neoplasia type 2.

In some aspects, the present invention is directed to methods of inhibiting cancer metastasis in a subject comprising administering to said subject and an effective amount of a tyrosine derivative.

In these aspects, an effective amount of a tyrosine derivative is an amount effective to inhibit cancer metastasis.

In this aspect, the term “inhibiting cancer metastasis” refers to reducing the number of cancer cells which depart the tissue or organ in which the cancer began (i.e., the primary tumor), and spread to another site within the subject's body.

In some embodiments, the cancer cells are non-small cell lung cancer brain cancer, appendix cancer, biliary cancer, choleangiocarcinoma, colon cancer, germ cell tumor, glioma, neuroblastoma, prostate cancer, tongue cancer, tonsil squamous cell carcinoma, urothelial cancer, adenoid cystic carcinoma, adrenal gland tumor, amyloidosis, anal cancer, ataxia-telangiectasia, Beckwith Wiedemann syndrome, bile duct cancer, Birt Hogg Dube syndrome, bladder cancer, bone cancer, brain tumor, breast cancer, breast cancer in men, carcinoid tumor, carney complex, cervical cancer, colorectal cancer, ductal carcinoma, endometrial cancer, esophageal cancer, gastric cancer, gastrontestinal stromal tumor—GIST, HER2-positive breast cancer, hereditary prostate cancer, islet cell tumor, juvenile polyposis syndrome, kidney cancer, laryngeal cancer, liver cancer, lobular carcinoma, lung cancer, lung cancer—small cell, malignant glioma, mastocytosis, melanoma, meningioma, multiple myeloma, myelodysplastic syndrome (MDS), nasopharyngeal cancer, neuroendocrine tumor, nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma (childhood), polycythemia vera, prostate cancer, renal cell cancer, retinoblastoma (childhood), salivary gland cancer, sarcoma, sarcoma—alveolar soft part and cardiac, sarcoma—Kaposi, skin cancer (non-melanoma), small bowel cancer, small intestine cancer, stomach cancer, testicular cancer, thymoma, thyroid cancer, Turcot syndrome, uterine (endometrial) cancer, vaginal cancer, Von-Hippel-Lindau syndrome, or Wilms' tumor (childhood).

In other aspects, the present invention also provides methods of reducing the number of circulating metastatic seed cells in a subject, comprising administering to the subject an effective amount of a tyrosine derivative.

In these aspects, an effective amount of a tyrosine derivative is an amount effective reduce the number of circulating metastatic seed cells.

As used herein, “circulating metastatic seed cells” refers to cancer cells that have departed a tumor and are circulating in the systemic circulation or in the lymphatic circulation.

Reduction in the number of circulating metastatic seed cells can be measured by comparing the number of circulating metastatic seed cells before administering the tyrosine derivative to the number of circulating metastatic seed cells after administering the tyrosine derivative. Methods of measuring circulating metastatic seed cells are known in the art.

In some embodiments, the circulating metastatic seed cells are in the systemic circulation.

In other embodiments, the circulating metastatic seed cells are in the lymphatic circulation.

In some embodiments, the circulating metastatic seed cells are cells of non-small cell lung cancer, brain cancer, appendix cancer, biliary cancer, choleangiocarcinoma, colon cancer, germ cell tumor, glioma, neuroblastoma, prostate cancer, tongue cancer, tonsil squamous cell carcinoma, urothelial cancer, adenoid cystic carcinoma, adrenal gland tumor, amyloidosis, anal cancer, ataxia-telangiectasia, Beckwith Wiedemann syndrome, bile duct cancer, Birt Hogg Dube syndrome, bladder cancer, bone cancer, brain tumor, breast cancer, breast cancer in men, carcinoid tumor, carney complex, cervical cancer, colorectal cancer, ductal carcinoma, endometrial cancer, esophageal cancer, gastric cancer, gastrontestinal stromal tumor—GIST, HER2-positive breast cancer, hereditary prostate cancer, islet cell tumor, juvenile polyposis syndrome, kidney cancer, laryngeal cancer, liver cancer, lobular carcinoma, lung cancer, lung cancer—small cell, malignant glioma, mastocytosis, melanoma, meningioma, multiple myeloma, myelodysplastic syndrome (MDS), nasopharyngeal cancer, neuroendocrine tumor, nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma (childhood), polycythemia vera, prostate cancer, renal cell cancer, retinoblastoma (childhood), salivary gland cancer, sarcoma, sarcoma—alveolar soft part and cardiac, sarcoma—Kaposi, skin cancer (non-melanoma), small bowel cancer, small intestine cancer, stomach cancer, testicular cancer, thymoma, thyroid cancer, Turcot syndrome, uterine (endometrial) cancer, vaginal cancer, Von-Hippel-Lindau syndrome, or Wilms' tumor (childhood).

In some aspects, the present invention is directed to methods of reducing the risk of developing cancer in a subject at increased risk of developing cancer as a result of exposure to a carcinogen or an oncovirus, comprising administering to said subject an effective amount of a tyrosine derivative.

In these aspects, an effective amount of a tyrosine derivative is an amount effective reduce the risk of developing cancer in a subject at increased risk of developing cancer as a result of exposure to a carcinogen or an oncovirus.

The reduction in the risk of developing cancer can be measured by comparing the subject's risk of developing cancer before administration of the tyrosine derivative with the subject's risk of developing cancer after administration of the tyrosine derivative.

As used herein, the term “carcinogen” refers to a chemical substance or radiation that promotes carcinogenesis, i.e., the formation of cancer. In some embodiments, the carcinogen is a chemical substance. In other embodiments, the carcinogen is radiation.

As used herein, the term “oncovirus” refers to a virus that can cause cancer. In some embodiments, the oncovirus is Hepatitis B (HBV), Hepatitis C (HCV), Human T-lymphotropic virus (HTLV), Human papillomaviruses (HPV), Kaposi's sarcoma-associated herpesvirus (HHV-8), Merkel cell polyomavirus (MCV), Epstein-Barr virus (EBV), or Human Immunodeficiency Virus (HIV).

In some embodiments, the cancer is non-small cell lung cancer, brain cancer, appendix cancer, biliary cancer, choleangiocarcinoma, colon cancer, germ cell tumor, glioma, neuroblastoma, prostate cancer, tongue cancer, tonsil squamous cell carcinoma, urothelial cancer, adenoid cystic carcinoma, adrenal gland tumor, amyloidosis, anal cancer, ataxia-telangiectasia, Beckwith Wiedemann syndrome, bile duct cancer, Birt Hogg Dube syndrome, bladder cancer, bone cancer, brain tumor, breast cancer, breast cancer in men, carcinoid tumor, carney complex, cervical cancer, colorectal cancer, ductal carcinoma, endometrial cancer, esophageal cancer, gastric cancer, gastrontestinal stromal tumor—GIST, HER2-positive breast cancer, hereditary prostate cancer, islet cell tumor, juvenile polyposis syndrome, kidney cancer, laryngeal cancer, liver cancer, lobular carcinoma, lung cancer, lung cancer—small cell, malignant glioma, mastocytosis, melanoma, meningioma, multiple myeloma, myelodysplastic syndrome (MDS), nasopharyngeal cancer, neuroendocrine tumor, nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma (childhood), polycythemia vera, prostate cancer, renal cell cancer, retinoblastoma (childhood), salivary gland cancer, sarcoma, sarcoma—alveolar soft part and cardiac, sarcoma—Kaposi, skin cancer (non-melanoma), small bowel cancer, small intestine cancer, stomach cancer, testicular cancer, thymoma, thyroid cancer, Turcot syndrome, uterine (endometrial) cancer, vaginal cancer, Von-Hippel-Lindau syndrome, or Wilms' tumor (childhood), leukemia—acute lymphoblastic leukemia, leukemia—acute myeloid aml, leukemia—adult, leukemia—childhood, leukemia—chronic lymphocytic—CLL, leukemia—chronic myeloid—CML, leukemia—acute lymphocytic (ALL), lymphoma—Hodgkin's, lymphoma—non-Hodgkin's, or multiple myeloma.

In all methods of the invention, the subject is administered a tyrosine derivative. In some embodiments, the tyrosine derivative is a tyrosine derivative. In some embodiments, the tyrosine derivative is one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OMe HCl, H-D-3,5-diiodo-tyr-OMe HCl, H-D-tyr-OMe HCl, D-tyrosine methyl ester hydrochloride, D-tyrosine-OMe HCl, methyl D-tyrosinate hydrochloride, HD-tyr-OMe.HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)—OSu, Fmoc-tyr(3-NO₂)—OH, α-methyl-D-tyrosine, α-methyl-L-tyrosine, α-methyl-DL-tyrosine, and C1-C12 alkylester salts of α-methyl-DL-tyrosine such as α-methyl-DL-tyrosine methyl ester hydrochloride.

In some embodiments of the methods of the invention, the tyrosine derivative is α-methyl-DL-tyrosine.

In some embodiments of the methods of the invention, the subject is administered 10-1000 mg/kg of the tyrosine derivative. For example, in some embodiments, the subject is administered about 10 mg/kg, about 15 mg/kg, about 20 mg/kg, about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 925 mg/kg, about 950 mg/kg, about 975 mg/kg, or about 1000 mg/kg of the tyrosine derivative.

In some embodiments, the subject is administered 300-900 mg of the tyrosine derivative. In some embodiments, the subject is administered about 325 mg of the tyrosine derivative. In other embodiments, the subject is administered about 900 mg of the tyrosine derivative.

In some embodiments, the methods of the invention further comprise administering to the subject at least one of melanin, a melanin promoter, or a combination thereof. Thus, melanin can be used, one or more melanin promoters can be used, and both melanin and one or more melanin promoters can be used (either in separate dosage forms or in the same dosage form). Melanin promoters according to the present invention are chemical compounds that increase the production and/or the activity of melanin. Increased melanin levels are believed to reduce inflammation (through, for example, suppression of TNF) and exclude the sequestered lymph system. Melanin is a photo catalyst, and can therefore promote chemical reactions that generate free radicals which, in turn, can become accessible to cancer cells. Representative melanin promoters are methoxsalen and melanotan II.

In some embodiments of the methods, the subject is administered melanin, methoxsalen, or melanotan II.

In some embodiments, the methods of the invention further comprise administering to the subject a p450 3A4 promoter. “Cytochrome p450 3A4” (which can be abbreviated as “p450 3A4”) is a member of the cytochrome p450 superfamily of enzymes, and is a mixed-function oxidase that is involved in the metabolism of xenobiotics in the body. It has the widest range of substrates of all of the cytochromes. The function of a p450 3A4 promoter in the methods of the invention is to increase the expression and/or the activity of p450 3A4. The increased p450 3A4 expression and/or activity is believed to reduce cortisone and estrogen levels in the patient. Additionally, the increased p450 3A4 expression and/or activity also slightly decreases blood pH, which is believed to help to preserve or enhance melanin activity. Representative p450 3A4 promoters are 5,5-diphenylhydantoin (sold commercially as, for example, Dilantin), valproic acid, and carbamazepine.

In some embodiments of the methods, the subject is administered 5,5-diphenylhydantoin (sold commercially as, for example, Dilantin), valproic acid, or carbamazepine.

In some embodiments, the methods of the invention further comprise administering to the subject a leucine aminopeptidase inhibitors (alternatively known as leucyl aminopeptidase inhibitors). Leucine aminopeptidases are enzymes that preferentially catalyze the hydrolysis of leucine residues at the N-terminus of peptides and/or proteins. Inhibiting the expression and/or activity of leucine aminopeptidases is believed to assist in tumor reabsorption by increasing cholesterol transport to the liver. Generally, it is believed that aminopeptidase inhibitors, deplete sensitive tumor cells of specific amino acids by preventing protein recycling, thus generating an antiproliferative effect. Representative leucine aminopeptidase inhibitors are N-[(2S,3R)-3-amino-2-hydroxy-4-phenylbutyryl]-L-leucine, and rapamycin.

In some embodiments of the methods, the subject is administered N-[(2S,3R)-3-amino-2-hydroxy-4-phenylbutyryl]-L-leucine, or rapamycin.

In some embodiments, the methods of the invention comprise administering to the subject a tyrosine derivative; at least one of melanin, a melanin promoter, or a combination thereof; a p450 3A4 promoter; and a leucine aminopeptidase inhibitor.

In some embodiments, the methods of the invention comprise administering to the subject a tyrosine derivative; at least one of melanin, a melanin promoter, or a combination thereof; at least one of 5,5-diphenylhydantoin, valproic acid, or carbamazepine; and a leucine aminopeptidase inhibitor

In some embodiments, the methods of the invention comprise administering to the subject a tyrosine derivative; at least one of melanin, a melanin promoter, or a combination thereof; at least one of 5,5-diphenylhydantoin, valproic acid, or carbamazepine; and at least one of N-[(2S,3R)-3-amino-2-hydroxy-4-phenylbutyryl]-L-leucine or rapamycin.

In some embodiments, the methods of the invention comprise administering to the subject α-methyl-DL-tyrosine; at least one of melanin, a melanin promoter, or a combination thereof, at least one of 5,5-diphenylhydantoin, valproic acid, or carbamazepine; and at least one of N-[(2S,3R)-3-amino-2-hydroxy-4-phenylbutyryl]-L-leucine or rapamycin.

In some embodiments of the methods of the invention, the subject is a human.

In some embodiments of the methods of the invention, the subject is a human female.

In some embodiments of the methods of the invention, the subject is a human male.

In the methods of the present invention, the tyrosine derivative may be administered to the subject through any suitable administration route, including for example, orally, perorally, nasally, subcutaneously, intravenously, intramuscularly, transdermally, vaginally, rectally or in any combination thereof.

In some embodiments, the tyrosine derivative is administered in a pharmaceutical composition comprising the tyrosine derivative and a pharmaceutically acceptable excipient. Pharmaceutically acceptable excipients are known to those skilled in the art. See, e.g., Handbook of Pharmaceutical Excipients (Rowe, ed.), Pharmaceutical Press; 6th Revised edition (Jul. 31, 2009).

### EXAMPLES

### Example 1

A study was conducted to evaluate the antitumor effect of an oral formulations of α-methyl-DL-tyrosine in athymic nude mice bearing HCT116 tumors.

Athymic nude mice (female, 6-7 weeks old) were purchased from Charles River Laboratories, USA. The mice were housed in a ventilated cage rack with HEAP filter system, with 12-hour light cycle at 21-24° C. (70-75° F.) and 40-60% humidity. The mice were fed with Rodent Diet 5001 (Lab Diet) consisting of: >23% protein; >4.5% fat; and >6% fiber. The mice were allowed free access to water (Distill water, 1 ppm CI).

The mice were acclimated for 5 days prior to the implantation of HCT116 cells at 1 million cells per mouse in 100 ul of 1×PBS containing 50% Matrigel.

At the completion of study, mice were euthanized with isoflurane followed by inhalation of carbon dioxide (CO2).

The HCT116 cells (human colon carcinoma) were purchased from ATCC, and expanded to sufficient number of cells for tumor cell implantation.

Solutions of α-methyl-DL-tyrosine at 16.2 mg/ml, 32.4 mg/ml, and 64.8 mg/ml in water were prepared

After an acclimation period of 5 days, fifty (50) athymic nude female mice were inoculated in the thigh/flank area with 1 million of HCT116 cells suspended in 100 μl 1×PBS containing 50% Matrigel.

Beginning at Day 6 post cell inoculation, the tumor volume was measured every day until the average tumor volume reached about 100 mm³ (Tumor Volume=length×width×width×0.5).

Forty-four (44) out of the fifty (50) mice within the range of 50-150 mm³ were selected and randomly grouped into 4 groups (n=11): a control group treated with water (Group 1) and three groups treated with α-methyl-DL-tyrosine at 81 mg/kg (Group 2), 162 mg/kg (Group 3), and 324 mg/kg (Group 4).

The HCT-116 tumor bearing mice were oral gavage (PO) dosed daily at 5 ml/kg with water (Group 1), α-methyl-DL-tyrosine at 81 mg/kg (Group 2), α-methyl-DL-tyrosine at 162 mg/kg (Group 3), and α-methyl-DL-tyrosine at 324 mg/kg (Group 4) for 28 days.

Clinical observations were made daily; tumor volume was measured every three to four days prior to dosing until the completion of study or when individual tumor volume exceeds 2000 mm³.

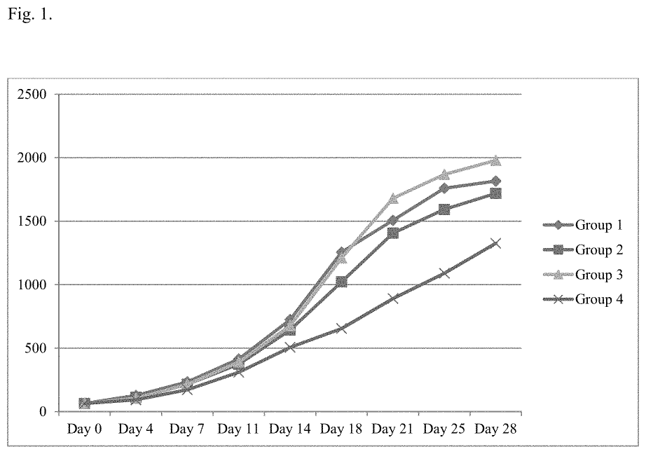

As summarized in Table 1 and presented in FIG. 1, the daily PO treatment of HCT-116 tumor bearing mice with α-methyl-DL-tyrosine at 324 mg/kg demonstrated a statistically significant (p<0.05, t-test) tumor growth inhibition.

The maximum anti-tumor efficacy was 48% reduction in tumor volume observed on Day 22.

TABLE 1
HCT-116 Tumor volume (mm³, group average) in treatment groups

| | | Day (post 1st dosing) | | | | | | | | |
|---|---|---|---|---|---|---|---|---|---|---|
| Group | | 0 | 4 | 7 | 11 | 14 | 18 | 21 | 25 | 28 |
| G1, water, n = 11 | Group Avg | 64.8 | 127.6 | 233.9 | 414.3 | 725.2 | 1255.8 | 1507.0 | 1759.5 | 1815.9 |
| G2, α-methyl-DL-tyrosine | Group Avg | 63.5 | 112.5 | 215.3 | 373.6 | 643.5 | 1022.3 | 1405.9 | 1592.4 | 1718.8 |
| at 162 mg/kg, n = 11 | | | | | | | | | | |
| G3, α-methyl-DL-tyrosine | Group Avg | 64.3 | 106.0 | 214.6 | 391.4 | 676.6 | 1210.3 | 1680.1 | 1868.0 | 1979.2 |
| at 81 mg/kg, n = 11 | | | | | | | | | | |
| G4, α-methyl-DL-tyrosine | Group Avg | 64.2 | 92.9 | 172.3 | 310.5 | 506.3 | 654.9 | 890.6 | 1090.1 | 1325.7 |
| at 324 mg/kg, n = 11 | | | | | | | | | | |

These data demonstrate that treatment of HCT116 tumor bearing mice with α-methyl-DL-tyrosine at 324 mg/kg daily resulted in a statistically significant (p<0.05, t-test) antitumor efficacy between Day 18 to Day 25. On Day 28, the tumor inhibition became non-significant simply because the study limited the maximum tumor volume at 2000 mm³ in the control group.

Moreover, in this study the α-methyl-DL-tyrosine was administered seven days after the tumor was implanted in the mouse. The fact that the α-methyl-DL-tyrosine inhibited growth of the tumor at the early phase of tumor implantation demonstrates that α-methyl-DL-tyrosine can inhibit metastatic conversion of precancerous cells.

### Example 2

During a routine colonoscopy, a patient is observed to have adenomatous polyps in the colon. The patient is administered 900 mg of α-methyl-DL-tyrosine, as four 225 mg tablets. The adenomatous polyps do not develop into colon cancer.

### Example 3

During a routine gynecological examination, a patient is observed to have cervical dysplasia (cervical intraepithelial neoplasm, CIN). The patient is administered 900 mg of α-methyl-DL-tyrosine, as four 225 mg tablets. The cervical dysplasia does not develop into cervical cancer.

### Example 4

During a routine physical examination, dysplastic moles are observed on a patient's skin. The patient is administered 900 mg of α-methyl-DL-tyrosine, as four 225 mg tablets. The dysplastic moles do not develop into melanoma.

### Example 5

A patient is exposed to ionizing radiation while receiving a diagnostic X-ray scan. The patient is administered 900 mg of α-methyl-DL-tyrosine, as four 225 mg tablets. The patient's risk of developing cancer as a result of the X-ray scan is reduced.

### Example 6

A chemical laboratory technician is inadvertently exposed to chloromethyl methyl ether (a known chemical carcinogen). The patient is administered 900 mg of α-methyl-DL-tyrosine, as four 225 mg tablets. The patient's risk of developing cancer as a result of the chemical exposure is reduced.

### Example 7

A patient is diagnosed as having been infected with the human papilloma virus (HPV; a known oncovirus). The patient is administered 900 mg of α-methyl-DL-tyrosine, as four 225 mg tablets. The patient's risk of developing cancer as a result of the HPV infection is reduced.

### Example 8

A report is made to governmental authorities regarding the detonation of a nuclear device. First-responders are dispatched to the area of the reported detonation, but are administered 900 mg of α-methyl-DL-tyrosine, as four 225 mg tablets. before being dispatched.

### Example 9

During a routine colonoscopy, a patient is observed to have adenomatous polyps in the colon. The patient is administered a combination of α-methyl-DL-tyrosine (900 mg of α-methyl-DL-tyrosine, as four 225 mg tablets); at least one of melanin, a melanin promoter, or a combination thereof; 5,5-diphenylhydantoin; and rapamycin. The adenomatous polyps do not develop into colon cancer.

### Example 10

During a routine gynecological examination, a patient is observed to have cervical dysplasia (cervical intraepithelial neoplasm, CIN). The patient is administered a combination of α-methyl-DL-tyrosine (900 mg of α-methyl-DL-tyrosine, as four 225 mg tablets); at least one of melanin, a melanin promoter, or a combination thereof; 5,5-diphenylhydantoin; and rapamycin. The cervical dysplasia does not develop into cervical cancer.

### Example 11

During a routine physical examination, dysplastic moles are observed on a patient's skin. The patient is administered a combination of α-methyl-DL-tyrosine (900 mg of α-methyl-DL-tyrosine, as four 225 mg tablets); at least one of melanin, a melanin promoter, or a combination thereof; 5,5-diphenylhydantoin; and rapamycin. The dysplastic moles do not develop into melanoma.

### Example 12

A patient is exposed to ionizing radiation while receiving a diagnostic X-ray scan. The patient is administered a combination of α-methyl-DL-tyrosine (900 mg of α-methyl-DL-tyrosine, as four 225 mg tablets); at least one of melanin, a melanin promoter, or a combination thereof; 5,5-diphenylhydantoin; and rapamycin. The patient's risk of developing cancer as a result of the X-ray scan is reduced.

### Example 13

A chemical laboratory technician is inadvertently exposed to chloromethyl methyl ether (a known chemical carcinogen). The patient is administered a combination of α-methyl-DL-tyrosine (900 mg of α-methyl-DL-tyrosine, as four 225 mg tablets); at least one of melanin, a melanin promoter, or a combination thereof; 5,5-diphenylhydantoin; and rapamycin. The patient's risk of developing cancer as a result of the chemical exposure is reduced.

### Example 14

A patient is diagnosed as having been infected with the human papilloma virus (HPV; a known oncovirus). The patient is administered a combination of α-methyl-DL-tyrosine (900 mg of α-methyl-DL-tyrosine, as four 225 mg tablets); at least one of melanin, a melanin promoter, or a combination thereof; 5,5-diphenylhydantoin; and rapamycin. The patient's risk of developing cancer as a result of the HPV infection is reduced.

### Example 15

A report is made to governmental authorities regarding the detonation of a nuclear device. First-responders are dispatched to the area of the reported detonation, but are administered a combination of α-methyl-DL-tyrosine (900 mg of α-methyl-DL-tyrosine, as four 225 mg tablets); at least one of melanin, a melanin promoter, or a combination thereof; 5,5-diphenylhydantoin; and rapamycin, before being dispatched.

In some embodiments, the disclosure is directed to the following aspects:
Aspect 1. A method of reducing the rate of tumor growth in a subject, comprising administering to the subject an effective amount of a tyrosine derivative.
Aspect 2. The method of aspect 1 wherein the tyrosine derivative is one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OMe HCl, H-D-3,5-diiodo-tyr-OMe HCl, H-D-tyr-OMe HCl, D-tyrosine methyl ester hydrochloride, D-tyrosine-OMe HCl, methyl D-tyrosinate hydrochloride, HD-tyr-OMe.HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)—OSu, Fmoc-tyr(3-NO₂)—OH, α-methyl-D-tyrosine, α-methyl-L-tyrosine, and α-methyl-DL-tyrosine.
Aspect 3. The method of aspect 1, wherein the tyrosine derivative is α-methyl-DL-tyrosine.
Aspect 4. The method of any one of aspects 1-3, wherein the tumor is a benign tumor.
Aspect 5. The method of aspect 4, wherein the benign tumor is a mass of precancerous cells.
Aspect 6. The method of aspect 4, wherein the benign tumor is a mass of precancerous skin cells.
Aspect 7. The method of aspect 4, wherein the benign tumor is a mass of precancerous cells of the respiratory tract, preferably, cells of the nasal cavity, paranasal sinuses, pharynx, larynx; trachea, bronchi, bronchioles, or lungs.
Aspect 8. The method of aspect 4, wherein the benign tumor is a mass of precancerous cells of the digestive tract, preferably, cells from mouth, throat; esophagus; stomach; small intestine; colon; rectum, or anus.
Aspect 9. The method of aspect 4, wherein the benign tumor is a mass of precancerous cells of the genitourinary tract organs, preferably, cells from kidney, bladder, prostate, testicles, uterus, fallopian tubes, ovaries, vagina, or external genitalia.
Aspect 10. The method of aspect 4, wherein the benign tumor is a mass of precancerous blood cells.
Aspect 11. The method of aspect 4, wherein the benign tumor is a mass of precancerous breast cells.
Aspect 12. The method of aspect 4, wherein the benign tumor is a mass of precancerous cells of the endocrine organs, preferably, cells of the hypothalamus; pineal gland; pituitary gland; thyroid; parathyroid; thymus; adrenal gland, or pancreas.
Aspect 13. The method of aspect 4, wherein the benign tumor is a mass of precancerous brain cells.
Aspect 14. The method of aspect 4, wherein the benign tumor is actinic keratosis, Bowen's disease; dyskeratosis congenita; ductal carcinoma in situ (breast); Sclerosing adenosis (breast); Small duct papilloma (breast); atypical lobular hyperplasia; oral submucous fibrosis; erythroplakia; lichen planus (oral); leukoplakia; proliferative verrucous leukoplakia; stomatitis nicotina; Barrett's esophagus; atrophic gastritis; adenomatous polyps in the colon; Plummer-Vinson syndrome (sideropenic dysphagia); hereditary nonpolyposis colorectal cancer (Lynch Syndrome); cervical dysplasia (cervical intraepithelial neoplasm, CIN); vaginal intraepithelial neoplasm (VAIN); anal dysplasia; lichen sclerosus; Bowen's disease (penile or vulvar); erythroplasia of Queyrat; bladder carcinoma in situ; monoclonal gammopathy of unknown significance; dysplastic moles; bronchial epithelial dysplasia; multiple endocrine neoplasia type 1, or multiple endocrine neoplasia type 2.
Aspect 15. The method of any one of aspects 1-3, wherein the tumor is malignant tumor.
Aspect 16. The method of aspect 15, wherein the malignant tumor is non-small cell lung cancer brain cancer, appendix cancer, biliary cancer, choleangiocarcinoma, colon cancer, germ cell tumor, glioma, neuroblastoma, prostate cancer, tongue cancer, tonsil squamous cell carcinoma, urothelial cancer, adenoid cystic carcinoma, adrenal gland tumor, amyloidosis, anal cancer, ataxia-telangiectasia, Beckwith Wiedemann syndrome, bile duct cancer, Birt Hogg Dube syndrome, bladder cancer, bone cancer, brain tumor, breast cancer, breast cancer in men, carcinoid tumor, carney complex, cervical cancer, colorectal cancer, ductal carcinoma, endometrial cancer, esophageal cancer, gastric cancer, gastrontestinal stromal tumor—GIST, HER2-positive breast cancer, hereditary prostate cancer, islet cell tumor, juvenile polyposis syndrome, kidney cancer, laryngeal cancer, liver cancer, lobular carcinoma, lung cancer, lung cancer—small cell, malignant glioma, mastocytosis, melanoma, meningioma, multiple myeloma, myelodysplastic syndrome (MDS), nasopharyngeal cancer, neuroendocrine tumor, nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma (childhood), polycythemia vera, prostate cancer, renal cell cancer, retinoblastoma (childhood), salivary gland cancer, sarcoma, sarcoma—alveolar soft part and cardiac, sarcoma—Kaposi, skin cancer (non-melanoma), small bowel cancer, small intestine cancer, stomach cancer, testicular cancer, thymoma, thyroid cancer, Turcot syndrome, uterine (endometrial) cancer, vaginal cancer, Von-Hippel-Lindau syndrome, or Wilms' tumor (childhood).
Aspect 17. A method of inhibiting malignant transformation of precancerous cells in a subject comprising administering to said subject an effective amount of a tyrosine derivative.
Aspect 18. The method of aspect 17 wherein the tyrosine derivative is one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OMe HCl, H-D-3,5-diiodo-tyr-OMe HCl, H-D-tyr-OMe HCl, D-tyrosine methyl ester hydrochloride, D-tyrosine-OMe HCl, methyl D-tyrosinate hydrochloride, HD-tyr-OMe.HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)—OSu, Fmoc-tyr(3-NO₂)—OH, α-methyl-D-tyrosine, α-methyl-L-tyrosine, and α-methyl-DL-tyrosine.
Aspect 19. The method of aspect 17, wherein the tyrosine derivative is α-methyl-DL-tyrosine. Aspect 20. The method of any one of aspects 17-19, wherein the precancerous cells are skin cells.
Aspect 21. The method of any one of aspects 17-19, wherein the precancerous cells are cells of the respiratory tract, preferably, cells of the nasal cavity, paranasal sinuses, pharynx, larynx; trachea, bronchi, bronchioles, or lungs.
Aspect 22. The method of any one of aspects 17-19, wherein the precancerous cells are cells of the digestive tract, preferably, cells from mouth, throat; esophagus; stomach; small intestine; colon; rectum, or anus.
Aspect 23. The method of any one of aspects 17-19, wherein the precancerous cells are cells of the genitourinary tract organs, preferably, cells from kidney, bladder, prostate, testicles, uterus, fallopian tubes, ovaries, vagina, or external genitalia.
Aspect 24. The method of any one of aspects 17-19, wherein the precancerous cells are blood cells.
Aspect 25. The method of any one of aspects 17-19, wherein the precancerous cells are breast cells.
Aspect 26. The method of any one of aspects 17-19, wherein the precancerous cells are cells of the endocrine organs, preferably, cells of the hypothalamus; pineal gland; pituitary gland; thyroid; parathyroid; thymus; adrenal gland, or pancreas.
Aspect 27. The method of any one of aspects 17-19, wherein the precancerous cells are brain cells.
Aspect 28. The method of any one of aspects 17-19, wherein the precancerous cells are actinic keratosis Bowen's disease; dyskeratosis congenita; ductal carcinoma in situ (breast); Sclerosing adenosis (breast); Small duct papilloma (breast); atypical lobular hyperplasia; oral submucous fibrosis; erythroplakia; lichen planus (oral); leukoplakia; proliferative verrucous leukoplakia; stomatitis nicotina; Barrett's esophagus; atrophic gastritis; adenomatous polyps in the colon; Plummer-Vinson syndrome (sideropenic dysphagia); hereditary nonpolyposis colorectal cancer (Lynch Syndrome); cervical dysplasia (cervical intraepithelial neoplasm, CIN); vaginal intraepithelial neoplasm (VAIN); anal dysplasia; lichen sclerosus; erythroplasia of Queyrat; bladder carcinoma in situ; monoclonal gammopathy of unknown significance; dysplastic moles; bronchial epithelial dysplasia; multiple endocrine neoplasia type 1, or multiple endocrine neoplasia type 2.
Aspect 29. The method of any one of aspects 17-19, wherein the precancerous cells are adenomatous polyps in the colon.
Aspect 30. A method of inhibiting cancer metastasis in a subject comprising administering to said subject and an effective amount of a tyrosine derivative.
Aspect 31. The method of aspect 30 wherein the tyrosine derivative is one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OMe HCl, H-D-3,5-diiodo-tyr-OMe HCl, H-D-tyr-OMe HCl, D-tyrosine methyl ester hydrochloride, D-tyrosine-OMe HCl, methyl D-tyrosinate hydrochloride, HD-tyr-OMe.HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)—OSu, Fmoc-tyr(3-NO₂)—OH, α-methyl-D-tyrosine, α-methyl-L-tyrosine, and α-methyl-DL-tyrosine.
Aspect 32. The method of aspect 31, wherein the tyrosine derivative is α-methyl-DL-tyrosine.
Aspect 33. The method of any one of aspects 30-32, wherein the cancer is non-small cell lung cancer brain cancer, appendix cancer, biliary cancer, choleangiocarcinoma, colon cancer, germ cell tumor, glioma, neuroblastoma, prostate cancer, tongue cancer, tonsil squamous cell carcinoma, urothelial cancer, adenoid cystic carcinoma, adrenal gland tumor, amyloidosis, anal cancer, ataxia-telangiectasia, Beckwith Wiedemann syndrome, bile duct cancer, Birt Hogg Dube syndrome, bladder cancer, bone cancer, brain tumor, breast cancer, breast cancer in men, carcinoid tumor, carney complex, cervical cancer, colorectal cancer, ductal carcinoma, endometrial cancer, esophageal cancer, gastric cancer, gastrontestinal stromal tumor—GIST, HER2-positive breast cancer, hereditary prostate cancer, islet cell tumor, juvenile polyposis syndrome, kidney cancer, laryngeal cancer, liver cancer, lobular carcinoma, lung cancer, lung cancer—small cell, malignant glioma, mastocytosis, melanoma, meningioma, multiple myeloma, myelodysplastic syndrome (MDS), nasopharyngeal cancer, neuroendocrine tumor, nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma (childhood), polycythemia vera, prostate cancer, renal cell cancer, retinoblastoma (childhood), salivary gland cancer, sarcoma, sarcoma—alveolar soft part and cardiac, sarcoma—Kaposi, skin cancer (non-melanoma), small bowel cancer, small intestine cancer, stomach cancer, testicular cancer, thymoma, thyroid cancer, Turcot syndrome, uterine (endometrial) cancer, vaginal cancer, Von-Hippel-Lindau syndrome, or Wilms' tumor (childhood).
Aspect 34. A method of reducing the number of circulating metastatic seed cells in a subject, comprising administering to the subject an effective amount of a tyrosine derivative.
Aspect 35. The method of aspect 34 wherein the tyrosine derivative is one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OMe HCl, H-D-3,5-diiodo-tyr-OMe HCl, H-D-tyr-OMe HCl, D-tyrosine methyl ester hydrochloride, D-tyrosine-OMe HCl, methyl D-tyrosinate hydrochloride, HD-tyr-OMe.HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)—OSu, Fmoc-tyr(3-NO₂)—OH, α-methyl-D-tyrosine, α-methyl-L-tyrosine, and α-methyl-DL-tyrosine.
Aspect 36. The method of aspect 34, wherein the tyrosine derivative is α-methyl-DL-tyrosine.
Aspect 37. The method of any one of aspects 34-36, wherein the circulating metastatic seed cells are in the systemic circulation.
Aspect 38. The method of any one of aspects 34-36, wherein the circulating metastatic seed cells are in the lymphatic circulation.
Aspect 39. The method of any one of aspects 34-36, wherein the circulating metastatic seed cells are cells of non-small cell lung cancer, brain cancer, appendix cancer, biliary cancer, choleangiocarcinoma, colon cancer, germ cell tumor, glioma, neuroblastoma, prostate cancer, tongue cancer, tonsil squamous cell carcinoma, urothelial cancer, adenoid cystic carcinoma, adrenal gland tumor, amyloidosis, anal cancer, ataxia-telangiectasia, Beckwith Wiedemann syndrome, bile duct cancer, Birt Hogg Dube syndrome, bladder cancer, bone cancer, brain tumor, breast cancer, breast cancer in men, carcinoid tumor, carney complex, cervical cancer, colorectal cancer, ductal carcinoma, endometrial cancer, esophageal cancer, gastric cancer, gastrontestinal stromal tumor—GIST, HER2-positive breast cancer, hereditary prostate cancer, islet cell tumor, juvenile polyposis syndrome, kidney cancer, laryngeal cancer, liver cancer, lobular carcinoma, lung cancer, lung cancer—small cell, malignant glioma, mastocytosis, melanoma, meningioma, multiple myeloma, myelodysplastic syndrome (MDS), nasopharyngeal cancer, neuroendocrine tumor, nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma (childhood), polycythemia vera, prostate cancer, renal cell cancer, retinoblastoma (childhood), salivary gland cancer, sarcoma, sarcoma—alveolar soft part and cardiac, sarcoma—Kaposi, skin cancer (non-melanoma), small bowel cancer, small intestine cancer, stomach cancer, testicular cancer, thymoma, thyroid cancer, Turcot syndrome, uterine (endometrial) cancer, vaginal cancer, Von-Hippel-Lindau syndrome, or Wilms' tumor (childhood).
Aspect 40. A method of reducing the risk of developing cancer in a subject at increased risk of developing cancer as a result of exposure to a carcinogen or an oncovirus, comprising administering to said subject an effective amount of a tyrosine derivative.
Aspect 41. The method of aspect 40 wherein the tyrosine derivative is one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OMe HCl, H-D-3,5-diiodo-tyr-OMe HCl, H-D-tyr-OMe HCl, D-tyrosine methyl ester hydrochloride, D-tyrosine-OMe HCl, methyl D-tyrosinate hydrochloride, HD-tyr-OMe.HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)—OSu, Fmoc-tyr(3-NO₂)—OH, α-methyl-D-tyrosine, α-methyl-L-tyrosine, and α-methyl-DL-tyrosine.
Aspect 42. The method of aspect 40, wherein the tyrosine derivative is α-methyl-DL-tyrosine.
Aspect 43. The method of any one of aspects 40-42, wherein the carcinogen is a chemical substance.
Aspect 44. The method of any one of aspects 40-42, wherein the carcinogen is radiation.
Aspect 45. The method of any one of aspects 40-42, wherein the oncovirus is Hepatitis B (HBV), Hepatitis C (HCV), Human T-lymphotropic virus (HTLV), Human papillomaviruses (HPV), Kaposi's sarcoma-associated herpesvirus (HHV-8), Merkel cell polyomavirus (MCV), Epstein-Barr virus (EBV), or Human Immunodeficiency Virus (HIV).
Aspect 46. The method of any one of aspects 40-42, wherein the cancer is non-small cell lung cancer, brain cancer, appendix cancer, biliary cancer, choleangiocarcinoma, colon cancer, germ cell tumor, glioma, neuroblastoma, prostate cancer, tongue cancer, tonsil squamous cell carcinoma, urothelial cancer, adenoid cystic carcinoma, adrenal gland tumor, amyloidosis, anal cancer, ataxia-telangiectasia, Beckwith Wiedemann syndrome, bile duct cancer, Birt Hogg Dube syndrome, bladder cancer, bone cancer, brain tumor, breast cancer, breast cancer in men, carcinoid tumor, carney complex, cervical cancer, colorectal cancer, ductal carcinoma, endometrial cancer, esophageal cancer, gastric cancer, gastrontestinal stromal tumor—GIST, HER2-positive breast cancer, hereditary prostate cancer, islet cell tumor, juvenile polyposis syndrome, kidney cancer, laryngeal cancer, liver cancer, lobular carcinoma, lung cancer, lung cancer—small cell, malignant glioma, mastocytosis, melanoma, meningioma, multiple myeloma, myelodysplastic syndrome (MDS), nasopharyngeal cancer, neuroendocrine tumor, nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma (childhood), polycythemia vera, prostate cancer, renal cell cancer, retinoblastoma (childhood), salivary gland cancer, sarcoma, sarcoma—alveolar soft part and cardiac, sarcoma—Kaposi, skin cancer (non-melanoma), small bowel cancer, small intestine cancer, stomach cancer, testicular cancer, thymoma, thyroid cancer, Turcot syndrome, uterine (endometrial) cancer, vaginal cancer, Von-Hippel-Lindau syndrome, or Wilms' tumor (childhood).
Aspect 47. The method of any one of the preceding aspects, wherein the subject is a human.
Aspect 48. The method of any one of the preceding aspects, wherein the tyrosine derivative is administered in a pharmaceutical composition comprising the tyrosine derivative and a pharmaceutically acceptable excipient.
Aspect 49. The method of any one of the preceding aspects, further comprising administering to the subject at least one of melanin, a melanin promoter, or a combination thereof.
Aspect 50. The method of any one of the preceding aspects, further comprising administering to the subject a p450 3A4 promoter.
Aspect 51. The method of aspect 50, wherein the p450 3A4 promoter is 5,5-diphenylhydantoin, valproic acid, or carbamazepine.
Aspect 52. The method of any one of the preceding aspects, further comprising administering to the subject a leucine aminopeptidase inhibitor.
Aspect 53. The method of aspect 52, wherein the leucine aminopeptidase inhibitor is N-[(2S,3R)-3-amino-2-hydroxy-4-phenylbutyryl]-L-leucine or rapamycin.

What is claimed:

1. A method comprising administering to a subject an amount of a tyrosine derivative that is effective to:
reduce the rate of tumor growth in the subject, or
inhibit malignant transformation of precancerous cells in the subject, or
inhibit cancer metastasis in the subject, or
reduce the number of circulating metastatic seed cells in the subject, or
reduce the risk of developing cancer in the subject, wherein the subject is at increased risk of developing cancer as a result of exposure to a carcinogen or an oncovirus.

2. The method of claim 1 wherein the tyrosine derivative is one or more of methyl (2R)-2-amino-3-(2-chloro-4 hydroxyphenyl) propanoate, D-tyrosine ethyl ester hydrochloride, methyl (2R)-2-amino-3-(2,6-dichloro-3,4-dimethoxyphenyl) propanoate H-D-Tyr(TBU)-allyl ester HCl, methyl (2R)-2-amino-3-(3-chloro-4,5-dimethoxyphenyl) propanoate, methyl (2R)-2-amino-3-(2-chloro-3-hydroxy-4-methoxyphenyl) propanoate, methyl (2R)-2-amino-3-(4-[(2-chloro-6-fluorophenyl) methoxy] phenyl) propanoate, methyl (2R)-2-amino-3-(2-chloro-3,4-dimethoxyphenyl) propanoate, methyl (2R)-2-amino-3-(3-chloro-5-fluoro-4-hydroxyphenyl) propanoate, diethyl 2-(acetylamino)-2-(4-[(2-chloro-6-fluorobenzyl) oxy] benzyl malonate, methyl (2R)-2-amino-3-(3-chloro-4-methoxyphenyl) propanoate, methyl (2R)-2-amino-3-(3-chloro-4-hydroxy-5-methoxyphenyl) propanoate, methyl (2R)-2-amino-3-(2,6-dichloro-3-hydroxy-4-methoxyphenyl) propanoate, methyl (2R)-2-amino-3-(3-chloro-4-hydroxyphenyl) propanoate, H-DL-tyr-OMe HCl, H-3,5-diiodo-tyr-OMe HCl, H-D-3,5-diiodo-tyr-OMe HCl, H-D-tyr-OMe HCl, D-tyrosine methyl ester hydrochloride, D-tyrosine-OMe HCl, methyl D-tyrosinate hydrochloride, HD-tyr-OMe.HCl, D-tyrosine methyl ester HCl, H-D-Tyr-OMe-HCl, (2R)-2-amino-3-(4-hydroxyphenyl) propionic acid, (2R)-2-amino-3-(4-hydroxyphenyl) methyl ester hydrochloride, methyl (2R)-2-amino-3-(4-hydroxyphenyl) propanoate hydrochloride, methyl (2R)-2-azanyl-3-(4-hydroxyphenyl) propanoate hydrochloride, 3-chloro-L-tyrosine, 3-nitro-L-tyrosine, 3-nitro-L-tyrosine ethyl ester hydrochloride, DL-m-tyrosine, DL-o-tyrosine, Boc-Tyr (3,5-I₂)—OSu, Fmoc-tyr(3-NO₂)—OH, α-methyl-D-tyrosine, α-methyl-L-tyrosine, and α-methyl-DL-tyrosine.

3. The method of claim 1, wherein the tyrosine derivative is α-methyl-DL-tyrosine.

4. The method of claim 1, wherein the tumor is a benign tumor.

5. The method of claim 4, wherein the benign tumor is a mass of precancerous cells.

6. The method of claim 4, wherein the benign tumor is a mass of precancerous skin cells.

7. The method of claim 4, wherein the benign tumor is a mass of precancerous cells of the respiratory tract, preferably, cells of the nasal cavity, paranasal sinuses, pharynx, larynx; trachea, bronchi, bronchioles, or lungs.

8. The method of claim 4, wherein the benign tumor is a mass of precancerous cells of the digestive tract, preferably, cells from mouth, throat; esophagus; stomach; small intestine; colon; rectum, or anus.

9. The method of claim 4, wherein the benign tumor is a mass of precancerous cells of the genitourinary tract organs, preferably, cells from kidney, bladder, prostate, testicles, uterus, fallopian tubes, ovaries, vagina, or external genitalia.

10. The method of claim 4, wherein the benign tumor is a mass of precancerous blood cells.

11. The method of claim 4, wherein the benign tumor is a mass of precancerous breast cells.

12. The method of claim 4, wherein the benign tumor is a mass of precancerous cells of the endocrine organs, preferably, cells of the hypothalamus; pineal gland; pituitary gland; thyroid; parathyroid; thymus; adrenal gland, or pancreas.

13. The method of claim 4, wherein the benign tumor is a mass of precancerous brain cells.

14. The method of claim 4, wherein the benign tumor is actinic keratosis, Bowen's disease; dyskeratosis congenita; ductal carcinoma in situ (breast); Sclerosing adenosis (breast); Small duct papilloma (breast); atypical lobular hyperplasia; oral submucous fibrosis; erythroplakia; lichen planus (oral); leukoplakia; proliferative verrucous leukoplakia; stomatitis nicotina; Barrett's esophagus; atrophic gastritis; adenomatous polyps in the colon; Plummer-Vinson syndrome (sideropenic dysphagia); hereditary nonpolyposis colorectal cancer (Lynch Syndrome); cervical dysplasia (cervical intraepithelial neoplasm, CIN); vaginal intraepithelial neoplasm (VAIN); anal dysplasia; lichen sclerosus; Bowen's disease (penile or vulvar); erythroplasia of Queyrat; bladder carcinoma in situ; monoclonal gammopathy of unknown significance; dysplastic moles; bronchial epithelial dysplasia; multiple endocrine neoplasia type 1, or multiple endocrine neoplasia type 2.

15. The method of claim 1, wherein the tumor is malignant tumor.

16. The method of claim 15, wherein the malignant tumor is non-small cell lung cancer brain cancer, appendix cancer, biliary cancer, choleangiocarcinoma, colon cancer, germ cell tumor, glioma, neuroblastoma, prostate cancer, tongue cancer, tonsil squamous cell carcinoma, urothelial cancer, adenoid cystic carcinoma, adrenal gland tumor, amyloidosis, anal cancer, ataxia-telangiectasia, Beckwith Wiedemann syndrome, bile duct cancer, Birt Hogg Dube syndrome, bladder cancer, bone cancer, brain tumor, breast cancer, breast cancer in men, carcinoid tumor, carney complex, cervical cancer, colorectal cancer, ductal carcinoma, endometrial cancer, esophageal cancer, gastric cancer, gastrontestinal stromal tumor—GIST, HER2-positive breast cancer, hereditary prostate cancer, islet cell tumor, juvenile polyposis syndrome, kidney cancer, laryngeal cancer, liver cancer, lobular carcinoma, lung cancer, lung cancer—small cell, malignant glioma, mastocytosis, melanoma, meningioma, multiple myeloma, myelodysplastic syndrome (MDS), nasopharyngeal cancer, neuroendocrine tumor, nevoid basal cell carcinoma syndrome, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, pleuropulmonary blastoma (childhood), polycythemia vera, prostate cancer, renal cell cancer, retinoblastoma (childhood), salivary gland cancer, sarcoma, sarcoma—alveolar soft part and cardiac, sarcoma—Kaposi, skin cancer (non-melanoma), small bowel cancer, small intestine cancer, stomach cancer, testicular cancer, thymoma, thyroid cancer, Turcot syndrome, uterine (endometrial) cancer, vaginal cancer, Von-Hippel-Lindau syndrome, or Wilms' tumor (childhood).

17. The method of claim 1, wherein the subject is a human.

18. The method of claim 1, wherein the tyrosine derivative is administered in a pharmaceutical composition comprising the tyrosine derivative and a pharmaceutically acceptable excipient.

19. The method of claim 1, further comprising administering to the subject at least one of melanin, a melanin promoter, or a combination thereof.

20. The method of claim 1, further comprising administering to the subject a p450 3A4 promoter.

21. The method of claim 20, wherein the p450 3A4 promoter is 5,5-diphenylhydantoin, valproic acid, or carbamazepine.

22. The method of claim 1, further comprising administering to the subject a leucine aminopeptidase inhibitor.

23. The method of claim 22, wherein the leucine aminopeptidase inhibitor is N-[(2S,3R)-3-amino-2-hydroxy-4-phenylbutyryl]-L-leucine or rapamycin.
